(19) United States
(12) Patent Application Publication
(10) Pub. No.: US 2005/0021114 A1
(43) Pub. Date: Jan. 27, 2005

(54) Hot moxibustion unit

(76) Inventor: Masayoshi Hidaka, Kyoto-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/494,516

(22) PCT Filed: Apr. 17, 2002
(86) PCT No.: PCT/JP02/03792

(30) Foreign Application Priority Data
Nov. 30, 2001 (JP) 2001-365936

(51) Int. Cl. A61F 7/00

(52) U.S. Cl. 607/112; 601/15; 607/114

(57) ABSTRACT

The present invention relates to a hot moxibustion unit provided with a heater which generates heat upon application of an electric current, the hot moxibustion unit comprising a heater (2) which generates heat upon application of an electric current, and a thermal mat (4) which is composed of a heat storage medium (3) which accumulates heat when heated by the heater (2) and a storage bag (8) which holds the heat storage medium (3), wherein the heater (2) is formed by attaching a flexible planar heating element (1) to a substrate sheet (6). This hot moxibustion unit closely fits a human body and provides increased comfort for the user.

### TECHNICAL FIELD

The present invention relates to a hot moxibustion unit comprising a heater that generates heat upon application of an electric current.

### BACKGROUND ART

A hot moxibustion unit of the above type achieves its thermal effects by heating a heat storage medium inside a thermal mat by means of a heater to provide warmth by the heat storage medium.

However, conventional hot moxibustion units have a drawback in that they use a hard-textured electric heater, which prevents them from providing a comfortable feel or closely fitting a human body.

The present invention was accomplished in view of the above-mentioned circumstances. It is, therefore, an object of the present invention to provide a hot moxibustion unit which allows a closer fit to a human body and enhances the user's comfort.

### DISCLOSURE OF THE INVENTION

In order to overcome the above-mentioned drawback, the present invention provides a hot moxibustion unit comprising a heater which generates heat upon application of an electric current, and a thermal mat which is composed of a heat storage medium which accumulates heat when heated by the heater and a storage bag which holds the heat storage medium, wherein the heater is formed by attaching a flexible planar heating element to a substrate sheet.

It is preferable that the planar heating element be in the form of a mesh woven with electrically conductive fine threads, and that the heater be formed by stitching the planar heating element to the substrate sheet.

It is preferable that the planar heating element be in the form of a fabric woven with electrically conductive fine threads.

It is preferable that the heat storage medium be formed of ceramic powder particles.

It is preferable that the heat storage medium be formed of a collection of randomly arranged heat-resistant fibers.

It is preferable that a heat-radiating aluminum layer be inserted between the planar heating element and the heat storage medium.

It is preferable that the storage bag be divided into multiple storage compartments, thus allowing the thermal mat to hold the heat storage medium in each of the storage compartments.

It is preferable that a pulse controller be provided which maintains the thermal mat at a constant temperature by pulse control of the planar heating element.

It is preferable that a thermo-controller be provided which is composed of a thermostat for controlling the power supply to the planar heating element and an operating temperature regulator for adjusting the operating temperature of the thermostat.

It is preferable that the planar heating element be disposed over a body-pressure-dispersing mat which is formed of a fluid substance, and that a thin heat storage medium be disposed over the planar heating element, the planar heating element being in the form of a mesh woven with electrically conductive fine threads, and the heat storage medium being formed of a collection of randomly arranged heat-resistant fibers.

It is preferable that the collection of randomly arranged fibers be a collection of randomly arranged carbon fibers.

### BRIEF DESCRIPTION OF THE DRAWINGS

FIG. 1 is a perspective view of a hot moxibustion unit according to the first embodiment of the present invention.

FIG. 2 is a sectional view taken along line X-X of FIG. 1.

FIG. 3 is a plan view of a heater of the hot moxibustion unit according to the present invention.

FIG. 4 is an enlarged sectional view of the hot moxibustion unit according to the present invention.

FIG. 5 shows the circuit configuration of a pulse controller of the hot moxibustion unit according to the present invention.

FIG. 6 shows the operating waveform for a pulse controller of the hot moxibustion unit according to the present invention.

FIG. 7 shows the operating waveform for a pulse controller of the hot moxibustion unit according to the present invention, and the corresponding temperature change profile for a thermal mat.

FIG. 8 shows the circuit configuration of a thermo-controller of the hot moxibustion unit according to the present invention.

FIG. 9 is a sectional view of a hot moxibustion unit according to the second embodiment of the present invention.

FIG. 10 is a sectional view of a hot moxibustion unit according to the third embodiment of the present invention.

### BEST MODES FOR CARRYING OUT THE INVENTION

Referring to the drawings, embodiments of the present invention will be described below.

First Embodiment

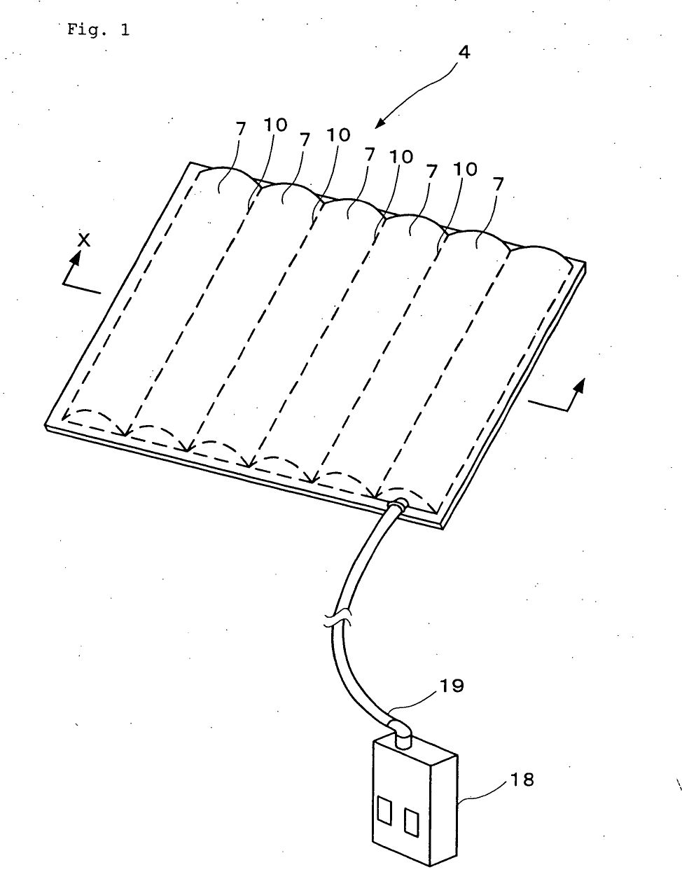

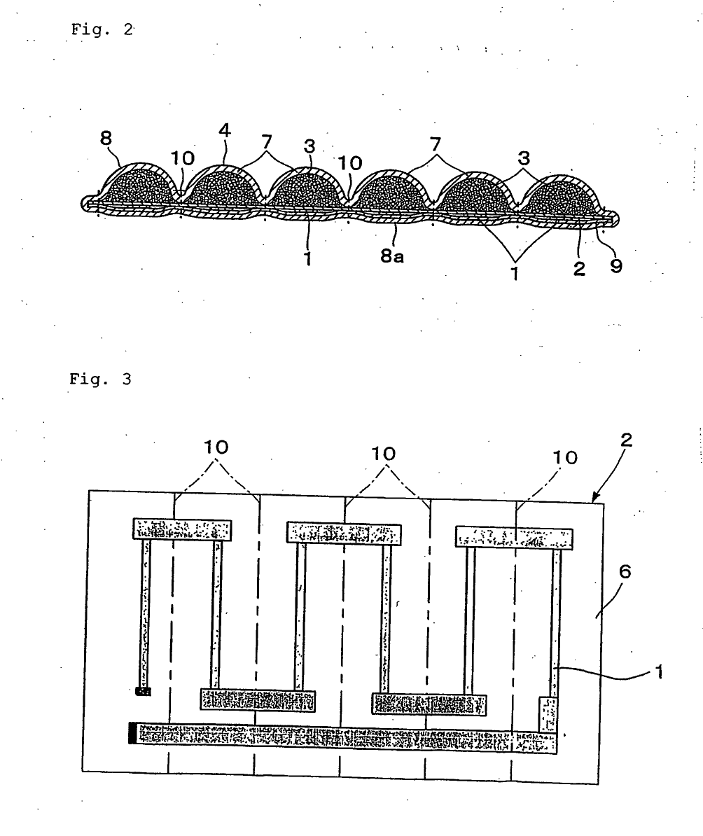

As shown in FIGS. 1 and 2, the hot moxibustion unit comprises a heater 2 incorporating a planar heating element 1, and a thermal mat 4 filled with a heat storage medium 3 which, when heated by the heater 2, accumulates heat and releases it to a human body to thereby provide the thermal effects of moxibustion.

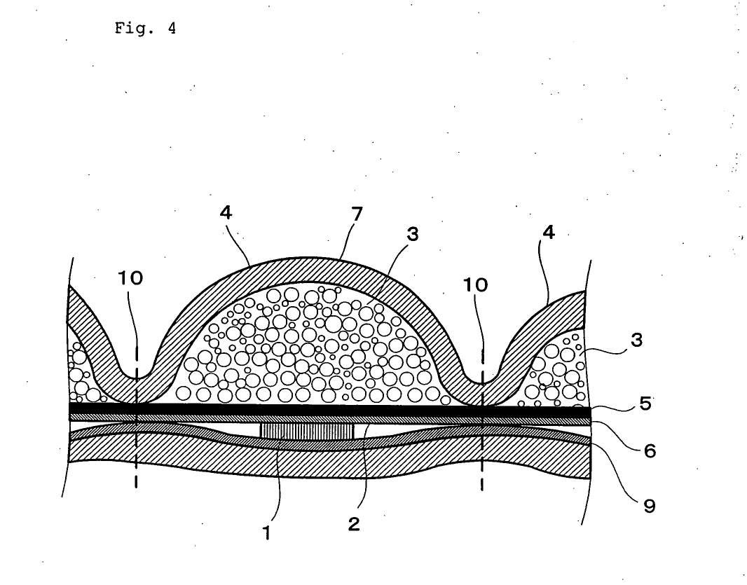

As shown in FIG. 4, the heater 2 is composed of a substrate sheet 6 and a planar heating element 1 which is stitched thereto with heat-resistant thread. The substrate sheet 6 is physically flexible, and has an aluminum layer 5 formed by vacuum deposition on one surface thereof, and the planar heating element 1 provided on the other surface thereof. Heat is released from the surface on which the aluminum layer 5 is formed.

The planar heating element 1 generates heat evenly throughout its plane by using a power supply that converts electric energy into thermal energy, and is formed of a fabric woven only with electrically conductive fine threads (i.e., a type of mesh construction), thus making it flexible.

The electrically conductive threads are made of conductive materials, such as stainless steel, and have a diameter of, for example, 20 μm to 500 μm, and preferably 200 μm to 300 μm. The open area ratio of the weave pattern (mesh open area ratio) is preferably about 50%.

The planar heating element 1, which is a fabric woven with electrically conductive fine threads, has high liquid permeability. The planar heating element 1 may be formed by connecting heating pieces made of the above fabric with each other by soldering, etc. The planar heating element 1 may also be formed by cutting the above fabric into a heating wiring pattern, thus eliminating the need to connect heating pieces.

The thermal mat 4 holds the heat storage medium 3 in a storage bag 8 that is divided into multiple storage compartments 7, the use of which allows the hot moxibustion unit to be attached to a human body so as to fit the shape of the body. The heater 2 is inserted between the heat storage medium 3 and a bag-forming sheet 8a which forms the rear surface of the storage bag 8, with the heat-radiating surface (aluminum layer 5) of the heater 2 being directed toward the heat storage medium 3.

As shown in FIGS. 2 and 4, a thermal insulation sheet 9 is sandwiched between the heater 2 and the bag-forming sheet 8a which forms the rear surface of the storage bag 8. The thermal insulation sheet 9 is made of a material having high strength, high elasticity and excellent insulation properties, such as aramid fiber.

As shown in FIGS. 1 and 2, the storage compartments 7 are formed by stitching the storage bag 8 so that the storage bag 8 is divided by stitch lines 10. The stitch lines 10 serve to secure the planar heating element 1 to the substrate sheet 6, as can be seen from FIG. 3. The planar heating element 1 is temporarily tacked to the substrate sheet 6 by threads, etc., prior to being properly secured by the stitch lines 10.

The heat storage medium 3 is formed of ceramic particles that can generate a large amount of far infrared rays. The heat storage medium 3, when heated by the planar heating element 1, generates far infrared rays, thereby inducing a temperature rise deep inside a human body, with the result of increasing the thermal effects of moxibustion, as well as providing a simple means of safety.

The ceramic powder particles have a particle diameter of, for example, about several tens of μm to about 10 mm, and preferably have superior heat-resistance and heat-retention properties as well as providing ease of use. Examples of the ceramic powder particles include synthetic (fired) and natural ceramic powder particles.

Fine ceramic powder particles of small diameter may be used, so that the thermal mat 4, when brought into contact with a human body, does not have a lumpy feel, and instead has a gentle feel on the skin. Such particles can be effectively used in treating symptoms such as menstrual pain, gastritis, etc. by the thermotherapeutic method, because the thermal mat 4 can be comfortably applied over the area where the pain is, for example, an abdominal area. The thermal mat 4 may also be made thin, so that it closely fits a human body in combination with the slimness and flexibility of the planar heating element 1, thus providing increased comfort to the body, and further allowing the hot moxibustion unit to be easily secured to the affected area using a supporter.

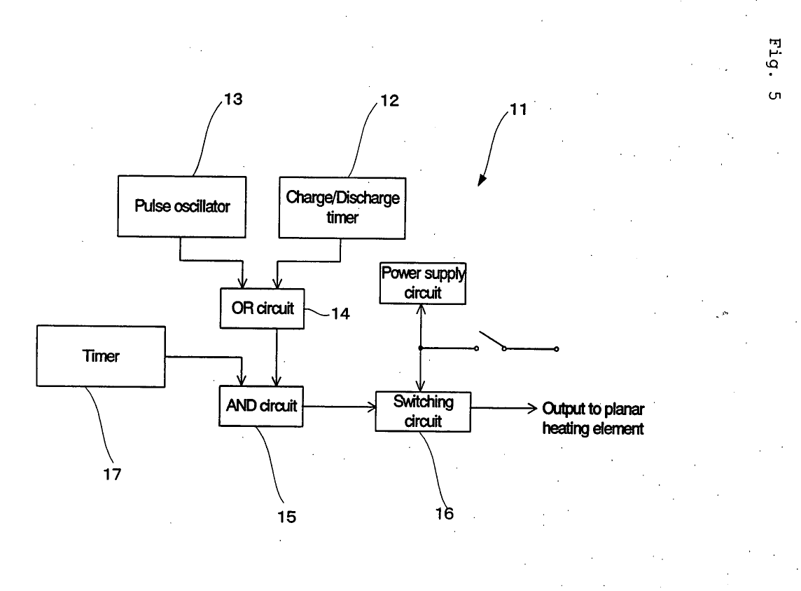

The hot moxibustion unit comprises a pulse controller 11 that maintains the thermal mat 4 at a constant temperature by pulse control. The pulse controller 11 comprises, as shown in FIG. 5, a charge/discharge timer 12, a pulse oscillator 13, an OR circuit 14, an AND circuit 15, a switching circuit 16, and a timer 17. Upon start-up, the planar heating element 1 is continuously energized using the charge/discharge timer 12 and, once the thermal mat 4 reaches a preset temperature, the planar heating element 1 is intermittently energized using the pulse oscillator 13 so that the above temperature is maintained. The planar heating element 1 is deenergized after a given period of time (e.g., 1 hour) using the timer 17. In the case of reenergizing the planar heating element 1 by the reset switch (not shown in the drawing) after a short period of deenergization, the planar heating element 1 is intermittently energized using the charge/discharge timer 12, instead of being continuously energized, thereby preventing the thermal mat 4 from becoming undesirably hot.

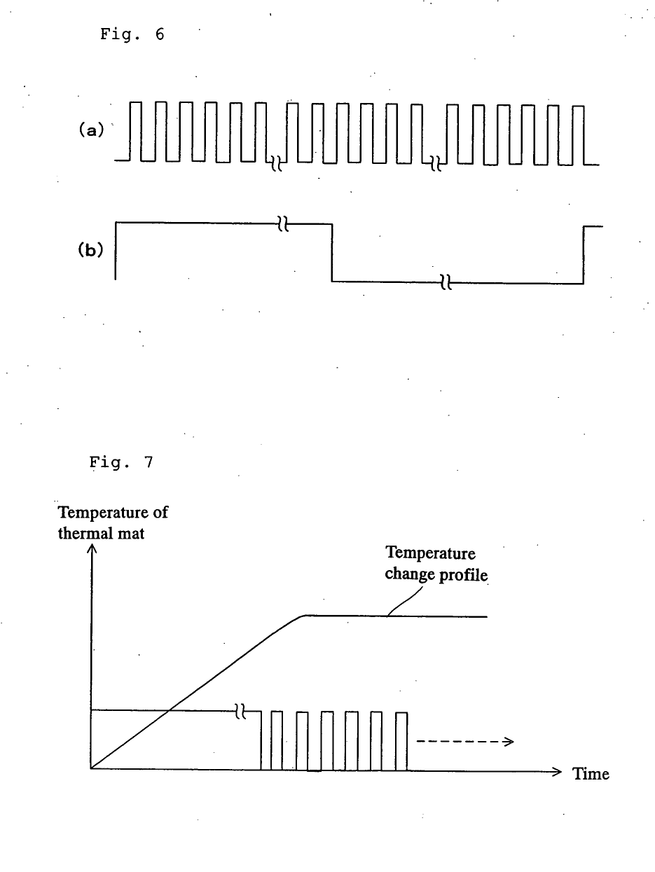

FIG. 6(a) shows the operating waveform for the pulse oscillator 13, and FIG. 6(b) shows the operating waveform for the charge/discharge timer 12. The pulse oscillator 13 repeats a control procedure in which the planar heating element 1 is, for example, energized for 0.04 second and then deenergized for 0.06 second. In contrast, the charge/discharge timer 12 allows the planar heating element 1 to be, for example, continuously energized for 15 to 20 minutes and then deenergized for 20 to 30 minutes. The OR circuit 14 receives the signals output from the pulse oscillator 13 and the charge/discharge timer 12, and outputs a high-level signal when the level of the signal provided from either the pulse oscillator 13 or the charge/discharge timer 12 is high. The AND circuit 15 receives the above high-level signal output from the OR circuit 14, and then outputs the same signal while the timer 17 outputs a high-level signal, thereby turning on/off the switching circuit 16. More specifically, by turning on the charge/discharge timer 12, the planar heating element 1 is continuously energized for a given period of time (15 to 20 minutes) until the thermal mat is heated to a temperature that would provide thermal stimulation. Thereafter, by turning on/off the pulse oscillator 13, the planar heating element 1 is intermittently energized so that it exhibits a desired heating pattern. As a result, a temperature change profile as shown in FIG. 7 is obtained which indicates that the thermal mat 4 is kept at a constant temperature after a given period of time (e.g., 15 to 20 minutes), thus providing stable thermal stimulation.

The AND circuit 15 receives the signals output from the OR circuit 14 and the timer 17, and outputs a high-level signal when the level of each of the signals provided from the OR circuit 14 and the timer 17 is high. The planar heating element 1 is deenergized using the timer 17 after, for example, 60 minutes of energizing in order to prevent troubles that may be caused by excessively prolonged operation, such as a low temperature burn.

As described above, the thermal mat 4 is maintained at a constant temperature by pulse control of the planar heating element 1. As shown in FIG. 1, the thermal mat 4 is connected, via an electrical cord 19, with an operator 18 having the pulse controller 11 built into its housing, thereby enabling remote control of the planar heating element 1. Accordingly, while the hot moxibustion unit is being held on a human body, the parts for controlling the planar heating element 1 are kept away from the body, thus providing increased comfort for the user.

The planar heating element 1 has superior liquid permeability, as mentioned earlier, and thus can be easily connected with wiring, circuit elements, etc., by soldering.

The hot moxibustion unit may draw its power from a low-voltage power source of 12 to 24 volts, an AC adaptor, batteries such as lithium batteries, or the like, and, therefore, may also be used when traveling or otherwise away from home.

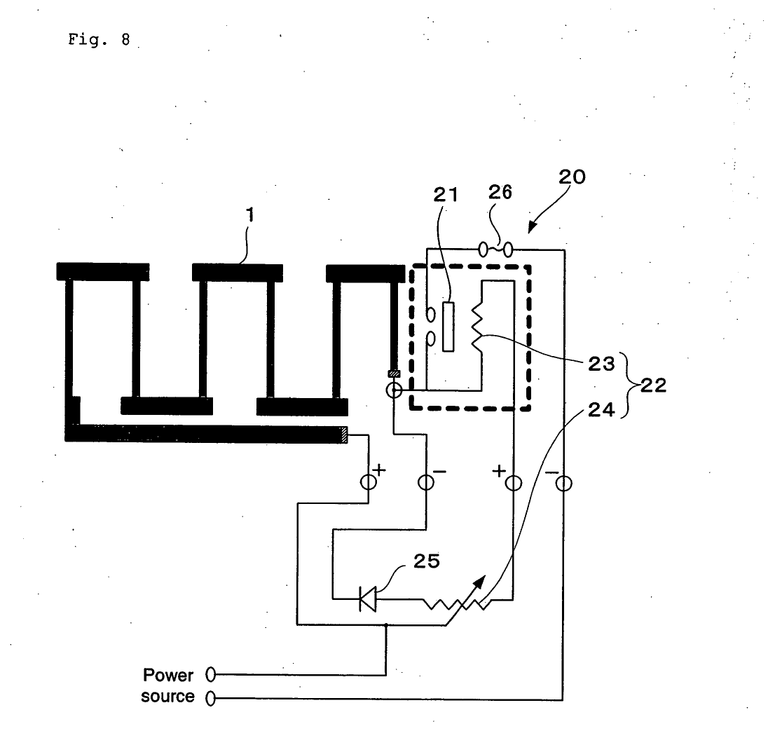

It is also possible to use a thermo-controller 20 as shown in FIG. 8 in place of the pulse controller 11. The thermo controller 20 comprises a thermostat 21 for controlling the power supply to the planar heating element 1, and an operating temperature regulator 22 for adjusting the operating temperature of the thermostat 21. When the hot moxibustion unit reaches a temperature of, for example, 70° C., the thermostat 21 turns off the power supply circuit for the planar heating element 1.

The operating temperature regulator 22 comprises a heat source 23 and a variable resistor 24 for controlling the current to be passed through the heat source 23. The heat source 23 heats the thermostat 21 to thereby adjust the temperature control range of the thermostat 21. In FIG. 8, reference numeral 25 indicates a light-emitting diode for monitoring the power supply status of the planar heating element 1, and reference numeral 26 indicates a thermal fuse.

Second Embodiment

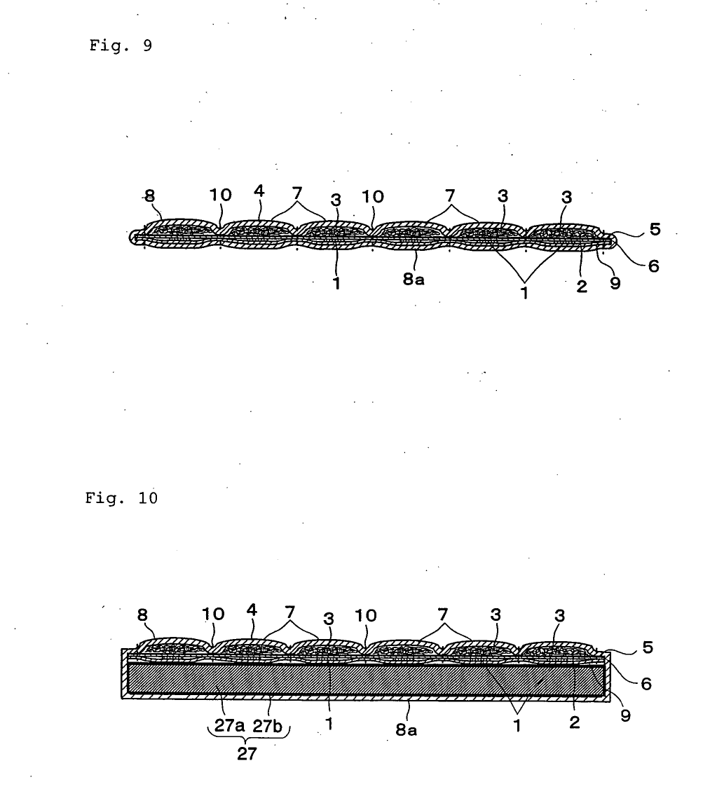

FIG. 9 illustrates the second embodiment of the present invention. A heat storage medium 3 is formed of a collection of randomly arranged carbon fibers. A collection of randomly arranged fibers refers to a fiber structure in which fibers are randomly assembled in a three-dimensional manner, as in the cotton padding of a futon mattress.

In the drawings, the structural elements that are equivalent or similar to those of the above embodiment are represented by the same reference numerals used for the embodiment, and the description of such elements is omitted.

In the hot moxibustion unit of the present embodiment, the heat radiated from a planar heating element 1 is first accumulated in the air trapped inside the collection of randomly arranged fibers, and then radiated from the air to a human body. In addition, since carbon fibers have high thermal conductivity, heat radiation through them allows thermal stimulation of the skin in a different manner from the above accumulated heat radiation. Two different types of thermal stimulation can be given to the affected area at the same time to further increase the thermal effects of moxibustion.

Also, the heat storage medium 3, which is a collection of randomly arranged fibers, may reduce the weight of the hot moxibustion unit, thus alleviating the burden on a human body.

Further, a thermal mat 4 is formed to have a planar shape as does the planar heating element 1, thus also allowing the overall hot moxibustion unit to have a planar shape, as shown in FIG. 9. Accordingly, the supporter that holds the hot moxibustion unit, when wrapped around a human body, allows the hot moxibustion unit to conform to the shape of the body, thus providing increased comfort to the body. In addition, the hot moxibustion unit is visually unobtrusive owing to its planar shape, and therefore can be readily used by women.

Furthermore, the hot moxibustion unit gives no unnecessary stimulation to the affected area of a human body, owing to the cushioning characteristics of the collection of randomly arranged fibers contained therein, and is suitably used for the treatment of symptoms such as menstrual pain, gastritis, etc.

In addition, the lifetime of the thermal mat is prolonged because of the excellent fatigue-resisting properties provided by the carbon fibers.

The collection of randomly arranged fibers may also be a collection of randomly arranged heat-resistant fibers, such as glass fibers, etc.

Further, the planar heating element 1 may be in the form of a mixed paper comprising electrically conductive fibers (such as carbon fibers), electrically non-conductive fibers and a binder, or a non-woven cloth formed with electrically conductive fibers (such as carbon fibers). The planar heating element 1 is secured to a substrate sheet by stitching or by using a heat-resistant adhesive, etc.

Third Embodiment

FIG. 10 illustrates the third embodiment of the present invention. The hot moxibustion unit comprises a body-pressure-dispersing mat 27, a heater 2 provided over the mat 27, and a heat storage medium 3 provided over the heater 2. The heater 2 is formed by a combination of a substrate sheet 6 and a planar heating element 1. The planar heating element 1 is made of a fabric woven only with electrically conductive fine threads. The heat storage medium 3 is formed of a collection of randomly arranged carbon fibers.

In the drawings, the structural elements that are equivalent or similar to those of the above embodiments are represented by the same reference numerals used for the embodiments, and the description of such elements is omitted.

The body-pressure-dispersing mat 27 is composed of a bag 27b and a fluid substance 27a stored therein, such as water, a gel product, etc. The body-pressure-dispersing mat 27, when brought into contact with a human body, deforms around the body in such a manner as to conform to the shape of the body. This increases the area in contact with the human body, allowing the mat to support the body not only locally, but over a wide contact area. The body-pressure-dispersing mat 27 thus disperses the body pressure throughout the mat and reduces the pressure that would be applied to the body, leading to the prevention of decubitus (bedsores). The mat returns to its original shape when no body pressure is put on the mat. Examples of the fluid substance 27a include gel products (e.g., visco-elastic urethane polymers, synthetic rubber containing mineral oil gel, or polymer gel), water, etc.

According to the present embodiment, the heater 2 is positioned between a human body and the body-pressure-dispersing mat 27, so that the cold touch typical of the fluid substance 27a of the body-pressure-dispersing mat 27 is not felt by the body, providing more comfortable use. Further, since the heat storage medium 3 is also positioned between the body and the body-pressure-dispersing mat 27, the collection of randomly arranged fibers constituting the heat storage medium 3 assures breathability, which cannot be provided by the fluid substance 27a.

Furthermore, the heater 2 and the heat storage medium 3 are thin and flexible, so that they can adapt to the deformation of the body-pressure-dispersing mat 27, without impairing the function of the body-pressure-dispersing mat 27. More specifically, the heat storage medium 3 and the planar heating element 1 are formed of a collection of randomly arranged carbon fibers and a fabric woven only with electrically conductive fine threads, respectively, and, therefore, can deform so as to conform to the shape of a human body along with the body-pressure-dispersing mat 27, thus maintaining the function of the mat 27 of allowing the body pressure to be dispersed by increasing the area in contact with the body.

The hot moxibustion unit of the present embodiment may be used as, for example, a floor cushion, bed mattress, chair seat, backrest, etc.

The hot moxibustion unit of the second or third embodiment also comprises the pulse controller or thermo-controller.

Configured as described above, the hot moxibustion unit of the present invention has many significant effects, which are described below.

As has been described earlier, in the hot moxibustion unit of the present invention, the heater is formed by attaching the flexible planar heating element to the substrate sheet. The hot moxibustion unit of the invention, therefore, does not have a lumpy feel, unlike a hard-textured electric heater, allowing it to fit more closely to a human body, and accordingly providing increased comfort to the body.

The planar heating element, when made of a mesh woven with flexible, electrically conductive fine threads, enables the following. While the planar heating element is being stitched to the substrate sheet, the sewing machine needle passes through the openings of the mesh, and the fine threads escape from the needle toward the openings adjacent to the opening through which the needle is passed, thus reducing the damage that might otherwise be caused to the planar heating element by the needle, with the result that the element is successfully stitched to the substrate sheet without being impaired. Therefore, the planar heating element may be secured to the substrate sheet without using a binder, thus requiring no time for curing the binder. This increases productivity as well as maintaining the quality of the planar heating element.

When the planar heating element is made of flexible fine threads, the whole element can be bent so as to conform to the shape of a human body, thus providing increased comfort to the body.

When the heat storage medium is formed of a collection of randomly arranged fibers, the weight of the hot moxibustion unit can be reduced, thus alleviating the burden to a human body. Further, the hot moxibustion unit can be naturally held on the body owing to the cushioning characteristics of the collection of randomly arranged fibers, providing added comfort.

When the heat storage medium is formed of a collection of randomly arranged carbon fibers, different types of stimulation can be given to the skin at the same time, further increasing the thermal effects of moxibustion.

When a heat-radiating aluminum layer is inserted between the planar heating element and the heat storage medium, the heat storage medium can be effectively heated owing to the heat-radiating capability of the aluminum layer.

When the heater is disposed over a body-pressure-dispersing mat which is formed of a fluid substance, and the heat storage medium is disposed over the heater, the planar heating element which constitutes the heater being made of a mesh woven with electrically conductive fine threads, and the heat storage medium being formed of a collection of randomly arranged heat-resistant fibers, the use of the body-pressure-dispersing mat and the excellent breathability of the collection of randomly arranged fibers serve to prevent bedsores effectively. Further, the cold touch of the body-pressure-dispersing mat can be offset by the heater.

1: A hot moxibustion unit comprising a heater which generates heat upon application of an electric current, and a thermal mat which is composed of a heat storage medium which accumulates heat when heated by the heater and a storage bag which holds the heat storage medium, wherein the heater is formed by attaching a flexible planar heating element to a substrate sheet.

2: A hot moxibustion unit according to claim 1, wherein the planar heating element is in the form of a mesh woven with electrically conductive fine threads, and the heater is formed by stitching the planar heating element to the substrate sheet.

3: A hot moxibustion unit according to claim 2, wherein the planar heating element is in the form of a fabric woven with electrically conductive fine threads, the electrically conductive fine threads having a diameter of 20 μm to 500 μm and a weave pattern with an open area ratio of about 50%.

4: A hot moxibustion unit according to claim 1, wherein the heat storage medium is formed of ceramic powder particles.

5: A hot moxibustion unit according to claim 1, wherein the heat storage medium is formed of a collection of randomly arranged heat-resistant fibers.

6: A hot moxibustion unit according to claim 1, wherein a heat-radiating aluminum layer is inserted between the planar heating element and the heat storage medium.

7: A hot moxibustion unit according to claim 1, wherein the storage bag is divided into multiple storage compartments, thus allowing the thermal mat to hold the heat storage medium in each of the storage compartments.

8: A hot moxibustion unit according to claim 1, wherein a pulse controller is provided which maintains the thermal mat at a constant temperature by pulse control of the planar heating element.

9: A hot moxibustion unit according to claim 1, wherein a thermo-controller is provided which is composed of a thermostat for controlling the power supply to the planar heating element and an operating temperature regulator for adjusting the operating temperature of the thermostat.

10: A hot moxibustion unit according to claim 1, wherein the planar heating element is disposed over a body-pressure-dispersing mat which is formed of a fluid substance, and a thin heat storage medium is disposed over the planar heating element, the planar heating element being in the form of a mesh woven with electrically conductive fine threads, and the heat storage medium being formed of a collection of randomly arranged heat-resistant fibers.

11: A hot moxibustion unit according to claim 5, wherein the collection of randomly arranged fibers is a collection of randomly arranged carbon fibers.

12: A method for manufacturing a hot moxibustion unit in which a planar heating element which generates heat upon application of an electric current is secured to a substrate sheet, and a heat storage medium which accumulates heat when heated by the planar heating element is stored in a storage bag, wherein:
the planar heating element is a fabric woven with electrically conductive fine threads having a diameter of 20 μm to 500 μm and a weave pattern with an open area ratio of about 50%;
bag-forming sheets which form the front and rear surfaces of the storage bag are stitched to each other with heat-resistant thread, thereby dividing the storage bag into multiple storage compartments and securing the planar heating element to the substrate sheet; and
the heat storage medium is stored in each of the storage compartments, thereby forming a thermal mat.

13: A hot moxibustion unit according to claim 10, wherein the collection of randomly arranged fibers is a collection of randomly arranged carbon fibers.
